(19) United States
(12) Patent Application Publication
(10) Pub. No.: US 2021/0022608 A1
(43) Pub. Date: Jan. 28, 2021

(54) Non-Contact Home-Tonometry System for Measuring Intraocular Pressure

(71) Applicant: IOPPRECEYESE LTD., Haifa (IL)

(72) Inventors: Niv Narkiss, Tel-Aviv (IL); Gil Shamai, Herzelia (IL); Iftach Kesten, Herzelia (US)

(21) Appl. No.: 16/980,448

(22) PCT Filed: Jan. 2, 2019
(86) PCT No.: PCT/IB2019/050019

§ 371 Date: Sep. 14, 2020

Related U.S. Application Data
(60) Provisional application No. 62/641,429, filed on Mar. 12, 2018.

(51) Int. Cl. A61B 3/16; A61F 9/00; H04N 13/207

(52) U.S. Cl. CPC A61B 3/165; A61B 2090/502; H04N 13/207; A61F 9/0026

(57) ABSTRACT

An ophthalmological analysis system, comprising: an air-puff generating device configured to apply an air-puff to a user's at least one eye; and at least one sensors board configured to detect and record deformation of a cornea of the user during the air-puff; wherein the detection is a three dimensional (3D) active stereo detection; the sensors board further configured to find two applanation points and hysteresis of the cornea and to calculate intraocular pressure in the user's at least one eye accordingly.

### CROSS-REFERENCE TO RELATED PATENT APPLICATIONS

This patent application claims priority from and is related to U.S. Provisional Patent Application Ser. No. 62641429, filed 12 Mar. 2018, this U.S. Provisional Patent Application incorporated by reference in its entirety herein.

### FIELD OF THE INVENTION

The present invention generally relates to medical devices and specifically to non-contact home-tonometry system for measuring intraocular pressure.

### BACKGROUND

Glaucoma is a disease that affects millions of people across the globe, about 3 million Americans suffer from this disease and 12 million more are at a risk of developing the disease. It is said to be the second leading cause of blindness and is correlated with an elevated intraocular pressure (IOP). In the standard model, the rise in intraocular pressure results when there is excessive aqueous humour in the anterior chamber of the eye because of the imbalance between the quantity of fluid secreted from the ciliary body and that drained through the trabecular meshwork. Since the chamber cannot increase in size, the fluid presses against the retina walls, compressing and damaging the cells along the optic nerve, causing the cells to die which leads to loss of vision.

A number of different tonometers have been developed over the years to measure IOP. Most of the existing tonometers, however, can only be used in clinical settings by health care professionals, such as ophthalmologists or optometrists. But, the IOP is not a constant value but fluctuates throughout the day with a 24-hour periodicity of circadian rhythms and hence necessitates measurement outside typical health care professional office hours. Accordingly, there is still a need for patient-operated tonometers that are easy to use and provide reliable IOP measurements.

Three basic principles are known for measuring the intraocular pressure, namely, impression tonometry, applanation tonometry and noncontact tonometry. The impression tonometer measures the depth of the indentability of the cornea caused by a metal stamp loaded with a known weight. For the same weight the indentability is inversely proportional to the intraocular pressure, that is the greater the indentability is, the lower is the intraocular pressure and conversely. A disadvantage with impression tonometry is that the placement of the tonometer and the impression of the metal stamp additionally increase the intraocular pressure so that the measured pressure does not correspond exactly to the actual intraocular pressure. Furthermore, the placement of the stamp on the cornea of the patient's eye is relatively stressful for the patients.

Furthermore, so-called applanation tonometers are also known for measuring the intraocular pressure, its measurement being based on application of the applanation principle. The applanation principle starts from lngbert's law which states that the pressure in a spherical container filled with liquid corresponds to the counter-pressure which flattens a certain surface of this sphere. The intraocular pressure can be measured on the basis of this law in two different ways. According to a first alternative, a tonometer with constant weight can be used and the flattened surface can be measured. According to an alternative method of measurement, the force required to flatten a known surface of constant size is used A Perkins applanation tonometer is known, which consists of a plastic cylinder whose lower planar end is provided with a gradation. A magnifying glass is located at the upper end. After instilling a fluorescent liquid into the conjunctival sac, the diameter of the applanated corneal surface can be determined by optical reading off on the gradation scale. In this case, the intraocular pressure is determined by means of a constant force.

In addition, an applanation tonometer operating on the principle of an applanated surface of constant size is known. In this case, the cornea is flattened using the quadrilateral base of a glass prism. The intraocular pressure is measured by intensifying the pressure of the prism on the eye until the flattened circular region of the cornea is at the same level as the four sides of the prism base. A disadvantage with applanation tonometers again is that as a result of the deformation of the cornea by means of an actuating element, considerable stress is produced for the patients.

So-called noncontact tonometers were developed to avoid this stressing produced by contact with a deforming tool. In these noncontact tonometers actuating devices are provided for deforming the cornea with which the cornea is deformed free from contact. For this purpose, a puff of compressed air is produced for example and directed onto the cornea. In known noncontact tonometers air puffs are directed onto the eye in the direction of the optic axis whereby the cornea is increasingly flattened and finally indented. To measure the deformation of the cornea, an obliquely incident bundle of parallel light rays is directed onto the cornea and the light reflected by the cornea is measured as a measurement signal. For this purpose, the reflected light can be intercepted by a light sensor, for example, where the light intensity measured by the light sensor varies as a function of the applanation of the cornea caused by the air flow.

A disadvantage in all known methods of measurement is that when measuring the intraocular pressure, the counter-pressure caused by the elastic deformation of the cornea is not taken into account. This is because the cornea itself is stretched over the vitreous body in the fashion of an elastic membrane so that during the measurement of the intraocular pressure a certain amount of force is required for its deformation which is included in the measurement results in a falsifying manner. This falsification is of a different magnitude in different patients since the properties of the cornea, especially its thickness and elasticity, vary within certain limits.

### SUMMARY OF THE INVENTION

According to an aspect of the present invention there is provided an ophthalmological analysis system, comprising: an air-puff generating device configured to apply an air-puff to a user's at least one eye; and at least one sensors board configured to detect and record deformation of a cornea of the user during the air-puff; wherein the detection is a three dimensional (3D) active stereo detection; the sensors board further configured to find two applanation points and hysteresis of the cornea and to calculate intraocular pressure in the user's at least one eye accordingly.

The air-puff generating device may comprise: a piston, activated by automatic drive means, slidably received by a cylinder housing and axially driven relative to the cylinder housing to compress air within a compression chamber defined by the cylinder housing; and an air discharge tube connected with the compression chamber for directing the air-puff along an optical axis towards the cornea.

The automatic drive means may comprise one of a linear motor, a rotary solenoid and a voice coil.

The air-puff generating device may further comprise a pressure sensor mounted inside the compression chamber.

The air-puff may be directed towards the cornea via an air-puff nozzle; wherein the air-puff generating device may further comprise a pressure sensor mounted inside the air-puff nozzle.

The air-puff generating device may comprise: a cylinder containing compressed air; an air chamber connected with the cylinder via an electric valve; and an air-puff nozzle connected with the air chamber via an electrical air regulator and an electrical valve.

The air-puff generating device may further comprise a pressure sensor mounted inside the air chamber.

The air-puff generating device may further comprise a pressure sensor mounted inside the air-puff nozzle; the pressure sensor may be configured to measure the air-puff pressure over time.

The system may further comprise an eye drop dispensing sub system comprising: an eye drop reservoir connected with an electric dosage pump; and a nozzle connected with the electric dosage pump; the eye drop dispensing sub system may be configured to perform an eye drop cycle thereby allowing an accurate dosage and placement of at least one drop into the user's at least one eye.

The eye drop reservoir may be configured to dispense the eye drops as one of spray and aerosol.

The at least one sensors board may comprise: a line-scan sensor; wherein the line-scan sensor is divided into two viewing angles thereby allowing calculation of medial displacement over time; an optical integrated lenses array mounted on top of the line-scan sensor; a pattern-projector; an air-puff nozzle; a multi-spectral Light Emitting Diode (LED); and a video camera.

The optical integrated lenses array may comprise four fixed mirrors.

The optical integrated lenses array may comprise two fixed mirrors and two movable mirrors.

According to another aspect of the present invention there is provided a method of calculating intraocular pressure, comprising: applying an air-puff to a user's at least one eye; detecting and recording, using a three dimensional (3D) active stereo detection, deformation of a cornea of the user during the air-puff by a line-scan sensor divided into two viewing angles thereby allowing calculation of medial displacement over time; finding two applanation points of the cornea; and calculating intraocular pressure in the user's at least one eye accordingly.

The applying an air-puff step may comprise: activating, by automatic drive means, a piston inside a compression chamber thereby discharging the air-puff along an optical axis towards a cornea of the user.

The automatic drive means may comprise one of a linear motor, a rotary solenoid and a voice coil.

The applying an air-puff step may comprise releasing a compressed air from a cylinder.

The method may further comprise: dispensing at least one eye drop into the user's at least one eye.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 shows a schematic drawing of the home-tonometry system according to embodiments of the present invention;

FIG. 2 shows a left-side view a patient wearing the head-mounted IOP unit, according to embodiments of the present invention;

FIG. 3 shows a top view of a patient wearing the head-mounted IOP unit, according to embodiments of the present invention;

FIG. 4 shows a section cut view of the left side of the head-mounted IOP unit, according to embodiments of the present invention;

FIG. 5 shows top and front views of the optical sensors board, according to embodiments of the present invention;

FIG. 6 shows a schematic view of the stereoscopic arrangement of the line-scan sensor, according to embodiments of the present invention;

FIG. 7 shows a schematic view of the stereoscopic arrangement of the line-scan sensor and the air-puff nozzle, according to embodiments of the present invention;

FIG. 8 shows a schematic view of the stereoscopic arrangement of the line-scan sensor and the rotating mirrors, according to embodiments of the present invention;

FIG. 9 shows front and side views of the pattern projector's Field of View (FOV) of the cornea, according to embodiments of the present invention;

FIG. 10 shows the components on the electronic-board and carrier and the projection of relevant components, according to embodiments of the present invention;

FIG. 11 shows a schematic view of an exemplary air puff generation sub system, according to embodiments of the present invention;

FIG. 12 shows a schematic view of an alternative exemplary air puff generation sub system, according to embodiments of the present invention;

FIG. 13 shows a schematic view of the exemplary air puff generation sub system of FIG. 12 with an eye drops dispensing sub system, according to embodiments of the present invention;

FIG. 14 shows an electrical block diagram of the system, according to embodiments of the present invention;

FIG. 15 shows an illustration of an eye subjected to an air-puff, according to embodiments of the present invention;

FIG. 16 shows an illustration of an eye in different deformation states during the air-puff stages, according to embodiments of the present invention;

FIG. 17 shows a schematic graph of the air-puff pressure and cornea curvature vs. time, according to embodiments of the present invention;

FIG. 18 is a flow chart showing the IOP measurement sequence carried out by system, according to embodiments of the present invention;

FIG. 19 is a flow chart showing the eye drop dispensing sequence, according to embodiments of the present invention; and

FIG. 20 shows the home-tonometry system and various uses.

### DETAILED DESCRIPTION OF THE INVENTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

The present invention provides an ophthalmological analysis system and method for measuring the Intra Ocular Pressure (IOP) of the eye.

The system of the present invention can easily be used outside the health professional's office, is non-invasive and measures the IOP using an optical system, so the need for anesthesia and the risk of infection are completely eliminated. With the system of the present invention, measurement of IOP can be done within fractions of a second, which eliminates the prolonged time required, in other devices, for positioning the patient before measurements can be done. Furthermore, the system of the present invention may measure both eyes simultaneously.

The measurement can be done in various positions, e.g., sitting, recumbence, lying down, etc. The accuracy of measurements with the disclosed system is not dependent on technique or the expertise of the operator. Accordingly, the system of the present invention is appropriate for use in non-clinical settings such as at a patient's home or in places across the globe where an ophthalmologic service is not readily available.

However, the system of the present invention can be used in clinical settings as well, and the invention as presently claimed should not be construed as limited to self-measurement devices or methods of self-measurement.

It is thus an objective of the present invention to provide a Non-Contact Tonometer (NCT) capable of measuring true IOP.

These and other objectives are achieved generally by a method of measuring intraocular pressure comprising the steps of:

A) Directing a air pulse at a cornea to cause reversible deformation of the cornea from an original state of convexity through a first state of applanation to a state of concavity, and back through a second state of applanation to the state of convexity;

B) Acquiring a first pressure value (P₁) associated with the air pulse at the time of the first state of applanation and a second pressure value (P₂) associated with the air pulse at the time of the second state of applanation; and

C) Calculating an intraocular pressure value using a predetermined function of the first pressure value (P₁) and the second pressure value (P₂), where the function was empirically derived to minimize cornea-related influence on the intraocular pressure value. According to embodiment of the present invention, the empirically derived function may be:

IOP=K₁(F₁×P₁+P₂)+K₂

Where—F₁, K₁ and K₂ are constants.

Both pressure values (P₁) and (P₂) are determined using image processing algorithms that find the two applanation points. The image processing algorithms are based on calculation of the cornea curvature index by using a high speed three-dimensional stereo camera images. The three-dimensional stereo camera optical arrangement may be passive or active, by using a pattern projector. The three-dimensional stereo camera optical arrangement uses a single line-sensor optically divided into two viewing angles, left and right, with a pattern projector. The algorithms analyze the pattern images from the two stereoscopic angles, left and right, and calculate the medial displacement over time. The optical system includes, among others, the three-dimensional stereo camera (line-scan sensor), a video camera, multispectral LEDs (visible and non-visible) and a pattern projector. The optical system is a self-calibrated system, with high field of view depth. According to embodiments of the present invention, the IOP system comprises two identical optical systems, one for each eye, left and right. The two optical systems have a mechanical apparatus for optimizing the distance between the two, left and right, corneas. Alternatively, the IOP system may comprise one optical system. In such a configuration, the system may further comprise a mechanical sub system for moving the optical system from one eye to the other.

The present invention includes an electrical subsystem with a board such as, for example, a System on Module (SOM) board that activates and controls the optical system, the air-puff compressor, and other components. In addition, the board is used for the image processing algorithms and IOP algorithms. Furthermore, the board communicates wirely or wirelessly with an external device such as a PC, smartphone, etc. running an IOP application.

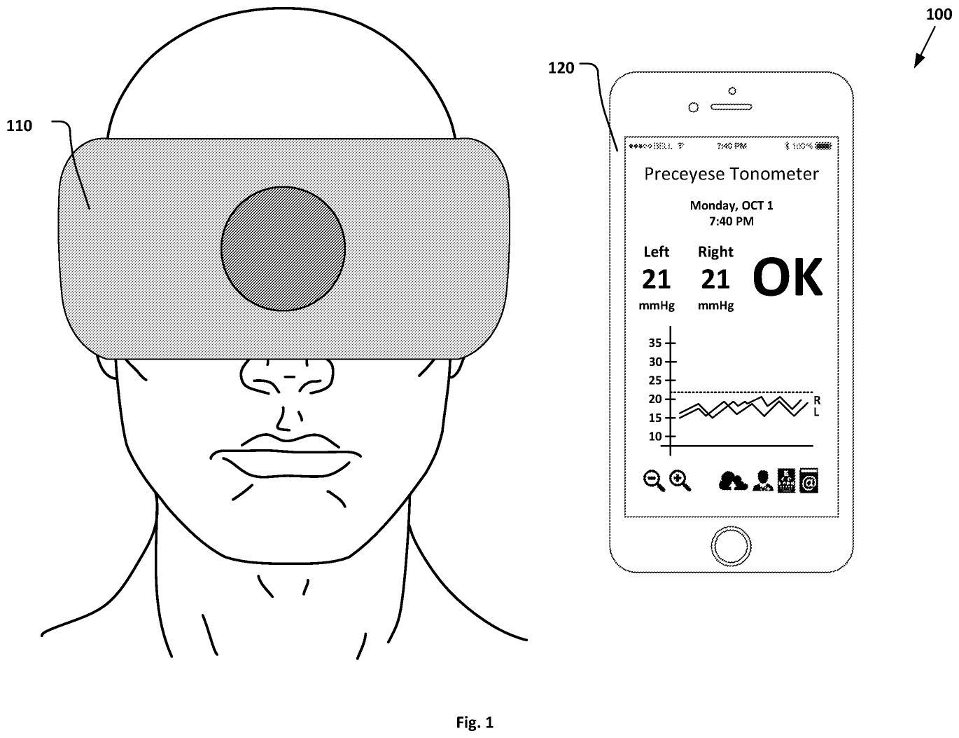

FIG. 1 shows a schematic view of the home-tonometry system 100 according to embodiments of the present invention, comprising: a head-mounted IOP unit 110 and an electronic communication device such as a PC, tablet, smartphone 120, etc. wirely or wirelessly connected with the head-mounted IOP unit 110. The electronic communication device runs a user application and serves as a display and interactive medium for communicating IOP measurement results to a database (e.g., a cloud base database) in order to allow ophthalmologist, general practitioner or others to view the IOP levels. It will be appreciated that the head-mounted unit 110 may be a fixed unit to which a user inserts his head.

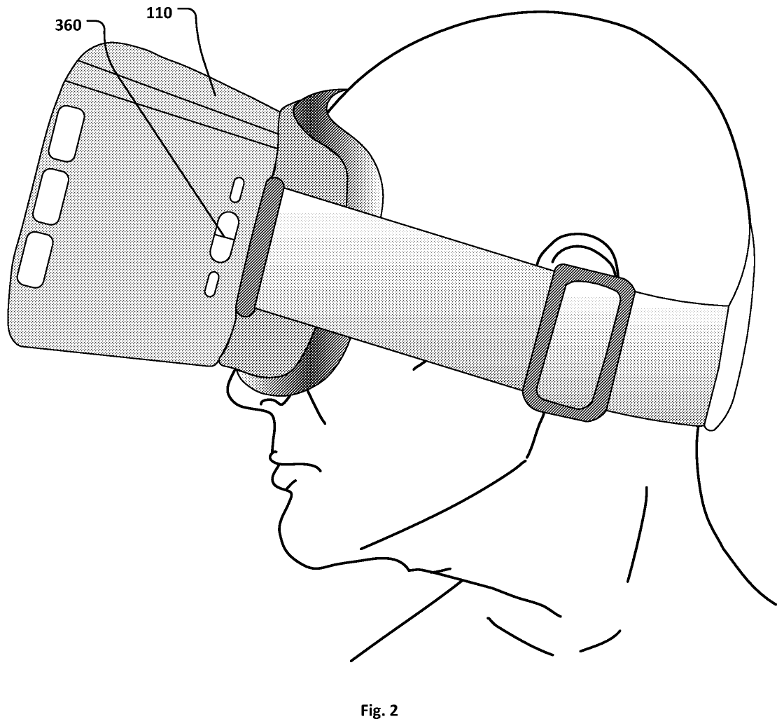

FIG. 2 shows a left side view of the head-mounted IOP unit 110, according to embodiments of the present invention. The head-mounted IOP unit 110 comprises a user interface (UI) panel and board 360 for one-hand easy operation, battery level, IOP level display status LEDs, a speaker for voice operational instructions, etc. It will be appreciated that the user interface (UI) 360 is not limited to these functionalities and/or features.

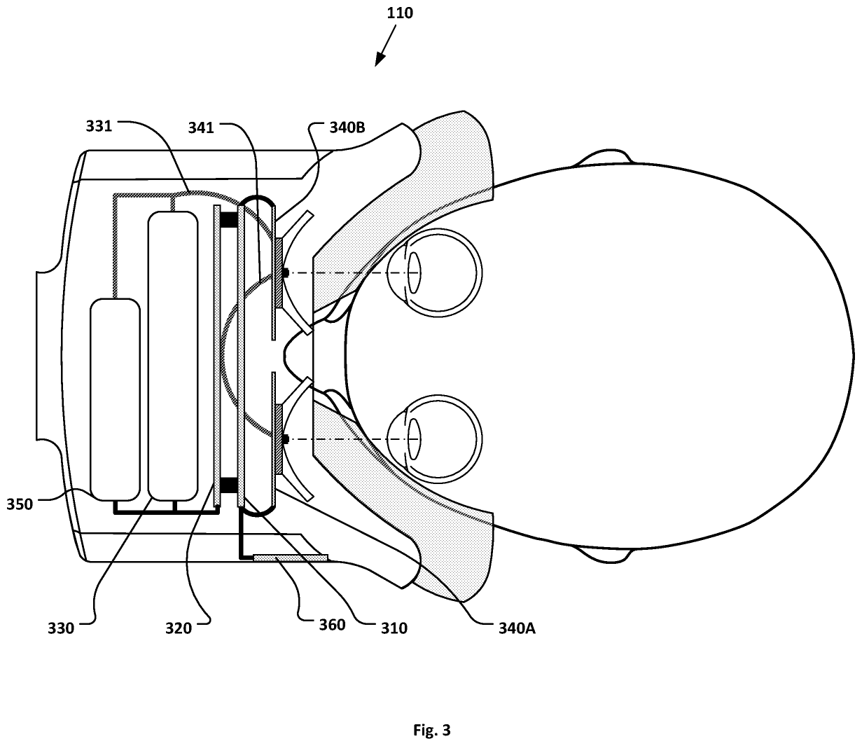

FIG. 3 shows a top view of the head-mounted IOP unit 110, according to embodiments of the present invention. The head-mounted IOP unit 110 further comprises a board, e.g., a System on Module (SOM) board 310 comprising a microcontroller and a field programmable gate array (FPGA) connected with a user interface (UI) panel and board 360. The user interface includes operation switches, LED indicators and a micro-display unit. A power board unit 320 comprising the power management of the system with a rechargeable battery that can be charged, for example, by a USB cable is connected with the board 310. The power board 320 comprises Wi-Fi and Bluetooth transceivers and is also connected with an air-puff generator system 330 and with an eye drop system 350. Two optical sensors boards 340A and 340B each located on the optical axis of the respective eye and connect with the board 310. The Air-puff system 330 dispenses the air puff to the optical sensors boards 340A and 340B via a tube 331. According to embodiments of the present invention, the air-puff dispensing tube 331 is connected to the optical sensors board 340B and the optical sensors board 340B is connected to the optical sensors board 340A via tube 341. The eye drop system 350 is connected with the air-puff generator 330.

It will be appreciated that the head-mount unit 110 is not limited to include two optical sensors boards. Alternatively, the unit 110 may include one optical sensors board which may move from one eye to the other for performing the process.

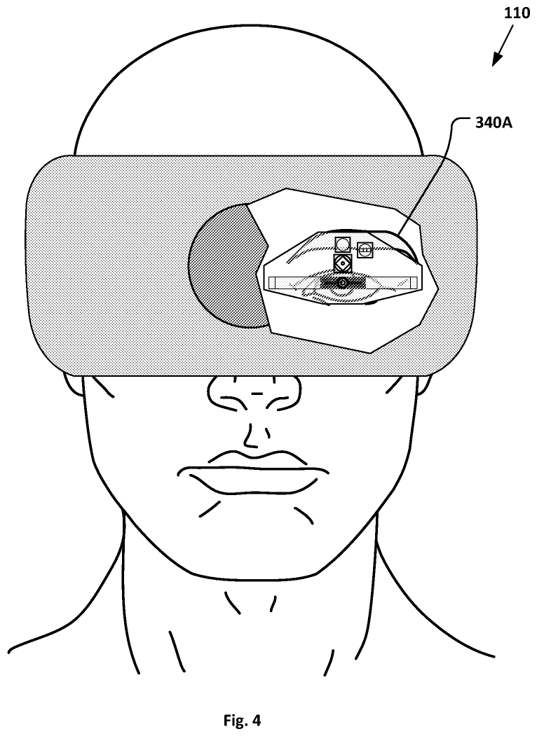

FIG. 4 shows a section cut view of the left side (right from the viewer perspective) of the head-mounted IOP unit 110 showing the optical sensor board 340A.

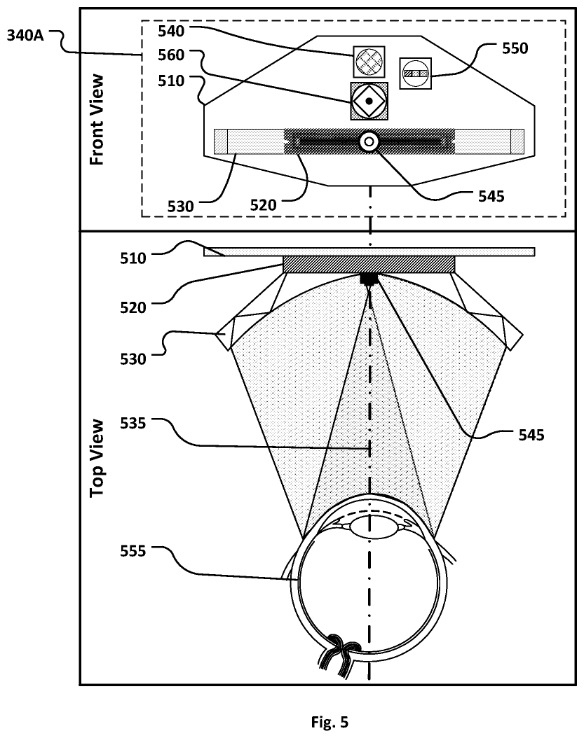

FIG. 5 shows top and front views of the optical sensor board 340A (or 340B), according to embodiments of the present invention. All the components of the optical sensor boards 340A and 340B are connected to an electronic-board and carrier 510. A high-speed and high-resolution line-scan sensor (black and white or color) 520 is mounted on the electronic-board and carrier 510. The line-scan sensor's focal plane array can be a Charge Coupled Device (CCD) or various types of Complementary Metal-Oxide-Semiconductor (CMOS). An optical integrated lenses array 530 is mounted on top of the line-scan sensor 520. The optical arrangement with respect to the optical axis 535 creates a stereo three-dimensional (3D) depth vision of the cornea with high-depth resolution of 1 micron. Moreover, with the pattern-projector 540, the optical arrangement is active stereo allowing superior depth resolution. The air-puff nozzle 545 is located in the dead zone of the optical stereoscopic arrangement and in-line with the optical axis 535. A multi-spectral Light Emitting Diode (LED) 550 allows illumination of the eye 555 with blue, green, red and near infra-red (NIR) wavelengths providing improved view of the cornea during the IOP measurement. In addition, a high-resolution video camera 560 enables a full view of the cornea. The camera 560 is the trigger for the IOP measurement and allows alignment of the eye 555 against the optical sensor board.

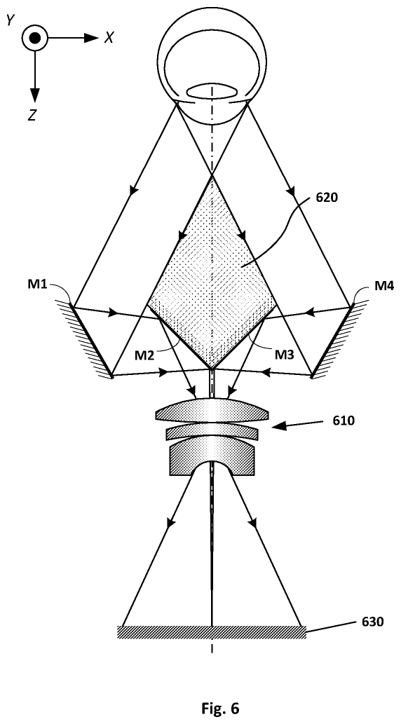

FIG. 6 shows a schematic view of the stereoscopic arrangement of the line-scan sensor, according to embodiments of the present invention. This stereoscopic arrangement uses one line scan sensor and the stereoscopic view is achieved by optically splitting the line-scan sensor into two segments. The lenses array 530 of FIG. 5 is represented by mirrors M1 to M4 and lens array 610. This optical arrangement provides better results than a stereoscopic optical arrangement with two different line-scan cameras. Area 620 represents the optical dead zone. Plane 630 represents the line-scan sensor plane.

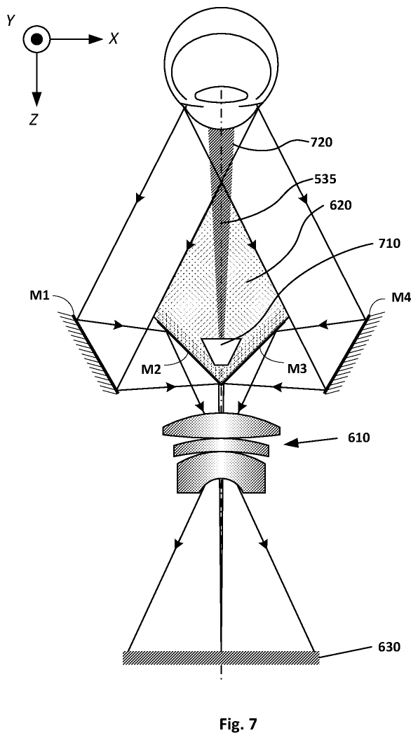

FIG. 7 shows a schematic view of the stereoscopic arrangement of the line-scan sensor with the air puff nozzle 710 in the center of the optical dead zone 620 and in-line with the optical axis 535, according to embodiments of the present invention. This opto-mechanical arrangement allows positioning the nozzle 710 on the optical axis 535 without blocking the view field. Beam 720 represents the air puff.

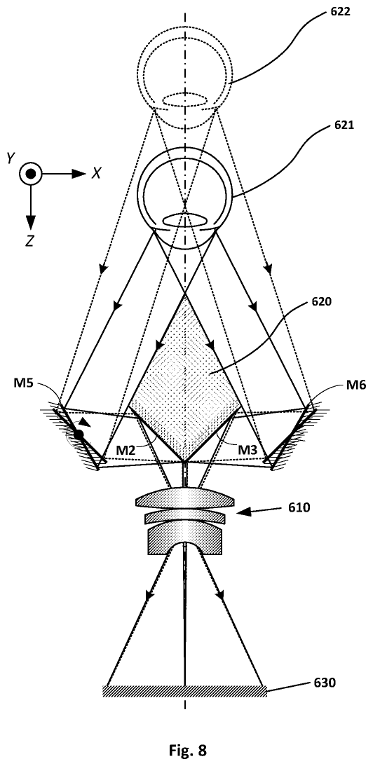

FIG. 8 shows a schematic view of the stereoscopic arrangement of the line-scan sensor with rotating mirrors M5 and M6 allowing different focusing depths, demonstrated by the eye 621 and the dashed eye 622. Mirrors M5 and M6 may rotate, for example, by using Micro Electro Mechanical System (MEMS).

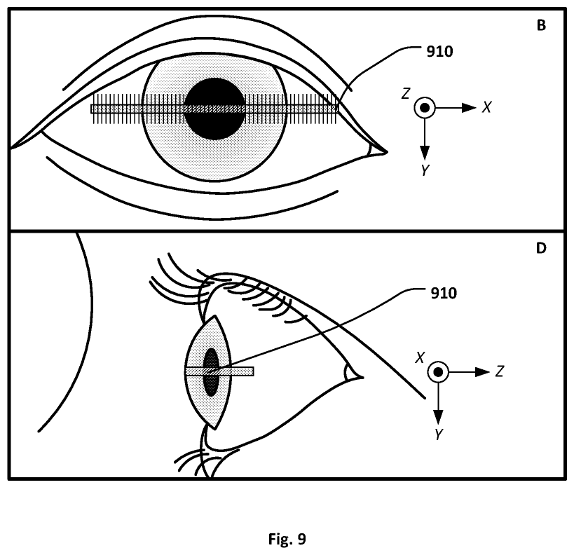

FIG. 9 shows front (B) and side (D) views of the pattern projector Field of View (FOV) 910 of the cornea. As can be seen, the FOV covers the cornea and part of the sclera thereby enabling a wide tolerance between the line-scan sensor and the cornea center.

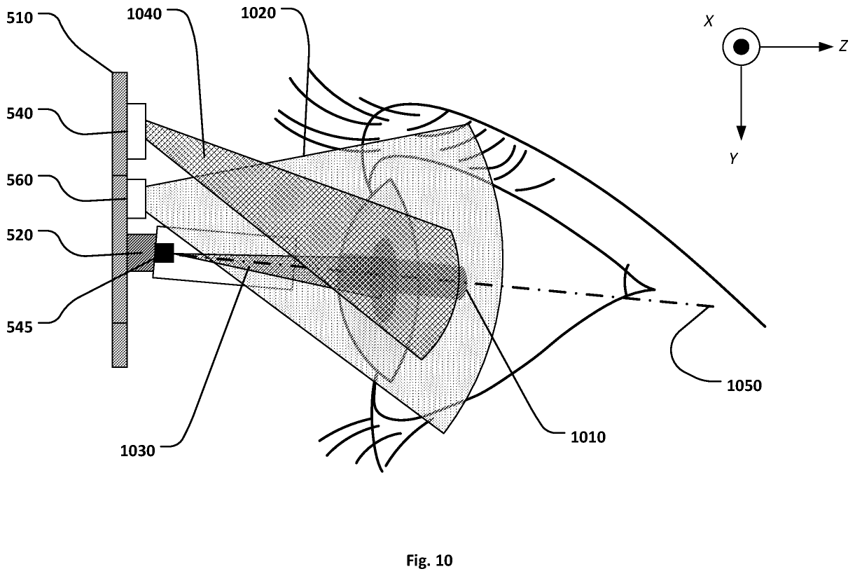

FIG. 10 shows the components on the electronic-board and carrier 510 and the projection of relevant components. The line-scan sensor 520 has a Field of View (FOV) 1010. The video camera 560 has a FOV 1020. The air puff-nuzzle 545 has an air-puff path 1030 with respect to the optical axis 1050 between the line-scan and the cornea center. The pattern projector 540 has a pattern FOV 1040. The video camera 560 allows the verification of the optical centration between the pupil and the center of the line-scan; the verification triggers the IOP measurement. The line-scan sensor measures the distance between the line-scan sensor 520 and the cornea for normalization of the exact air puff pressure upon the cornea in each measurement cycle. This eliminates Z axis calibration.

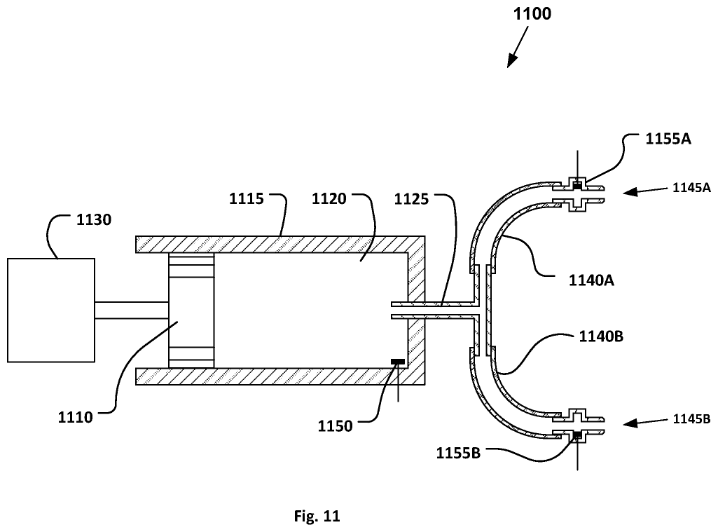

FIG. 11 shows a schematic view of an exemplary air puff generation sub system 1100 comprising air-puff compressor and distribution system, according to embodiments of the present invention. In this configuration, the air pulse is generated by a piston 1110 slidably received by a cylinder housing 1115 and axially driven relative to the cylinder housing to compress air within a compression chamber 1120 defined by the cylinder housing 1115. The compression chamber 1120 is connected with an air discharge tube 1125 for directing an air pulse along the optical axis towards the cornea. The piston 1110 is typically driven by automatic drive means 1130, for example, a linear motor, a rotary solenoid or a voice coil connected with the piston. The discharge tube 1125 splits into two tubes, 1140A for the left nozzle 1145A and 1140B for the right nozzle 1145B. The air pressure in the compression chamber 1120 is measured and monitored by a pressure sensor 1150. The pressure in the left and right nozzles 1145A and 1145B is measured and monitored by pressure sensors 1155A and 1155B.

Piston 1110 is pushed very rapidly from a starting position in order to generate a short air puff and moves back in a reciprocal direction to its starting position.

According to embodiments of the present invention, each nozzle, 1145A or 11458, may be closed in order to direct the air-puff to one of the eyes.

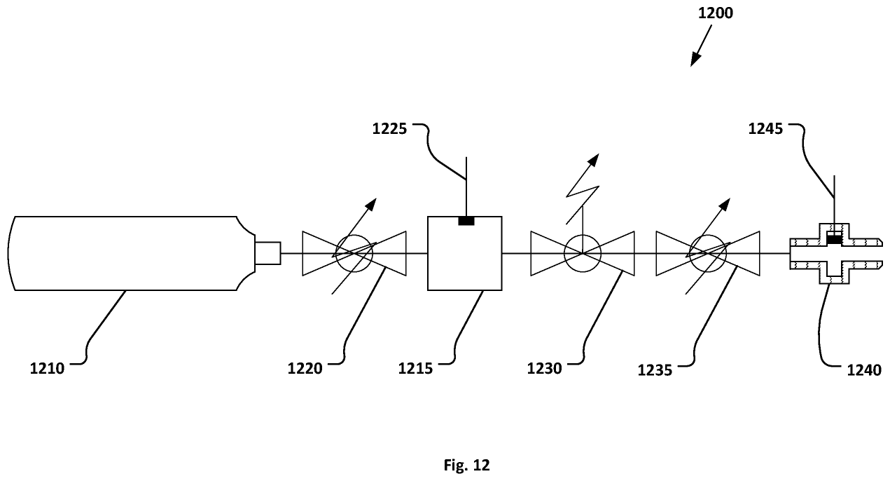

FIG. 12 shows a schematic view of an alternative exemplary air puff generation sub system 1200 comprising compressed air cylinder system and distribution system, according to embodiments of the present invention. Using a micro cylinder 1210 containing compressed air eliminates the use of a piston with moving parts. This allows a more robust system, allowing high optical measurement accuracy without the mechanical movement of a piston. In this configuration, the compressed air-cylinder 1210 is connected with the air chamber 1215 via an electric valve 1220. Air-chamber 1215 comprises a pressure sensor 1225 for measuring and regulating the pressure in the Air-chamber 1215 by using a closed loop control circuit with the electric valve 1220. According to embodiments of the present invention, the air pressure in the compressed air cylinder 1210 can be up to 250 atmospheres. The air pressure in the air-chamber 1215 can be up to 120 mmHg. The air chamber 1215 is connected with the air-puff nozzle 1240 via an electrical air regulator 1230 and an electrical valve 1235. An air pressure sensor 1245 is located in the nozzle 1240 and measures the air puff pressure over time during the IOP measurement.

According to embodiments of the present invention, the air chamber 1215 may be connected with another air-puff nozzle (not shown) for the other eye.

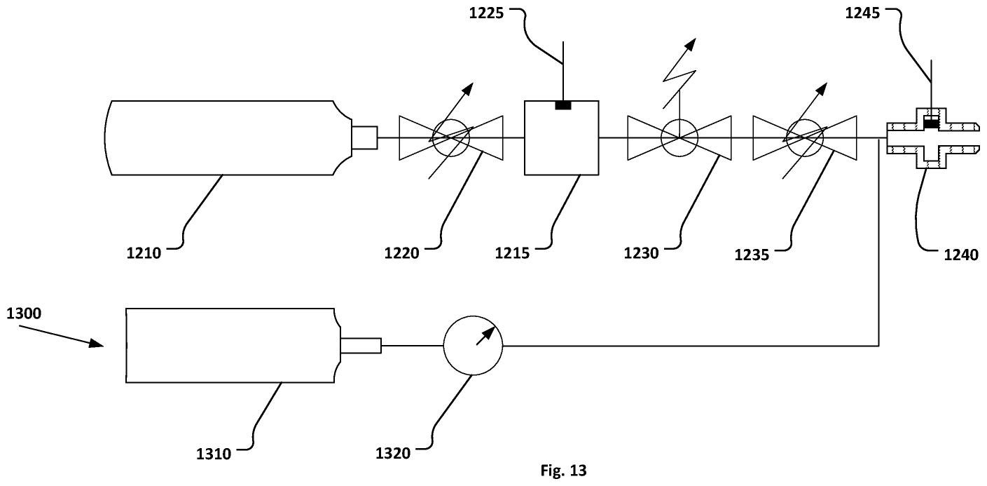

FIG. 13 shows a schematic view of the exemplary air puff generation sub system of FIG. 12 with an eye drop dispensing sub system 1300. An eye drop reservoir 1310 is connected with an electric dosage pump 1320 which is connected with the air-puff nozzle 1240. According to embodiments of the present invention, the system can perform an eye drop cycle, allowing an accurate dosage and placement of the drop(s) in the eye ball. According to embodiments of the present invention, the eye drop can be dispensed as a spray or aerosol. It will be appreciated that according to embodiments of the present invention, the eye drop dispensing sub system 1300 may be connected with the air puff generation sub system 1100 of FIG. 11.

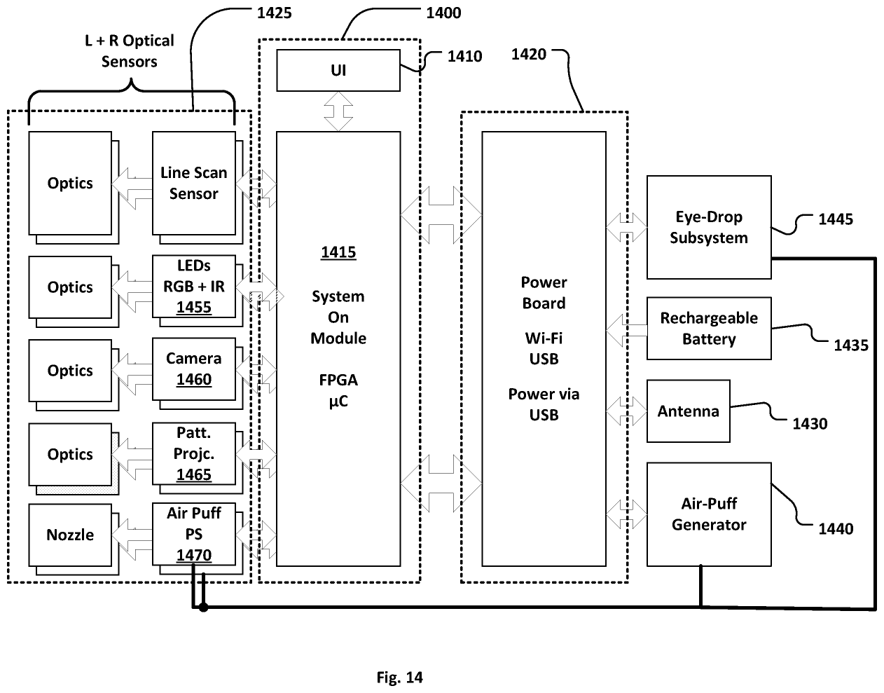

FIG. 14 shows the electrical block diagram of the system, according to embodiments of the present invention. The system processing board 1400 comprises a microcontroller with an FPGA 1415 and a User Interface (UI) 1410. The system processing board 1400 is connected with the power board 1420 and the two identical left and right optical sensor boards 1425. The power board 1420 supplies the power to the components of the system using a rechargeable battery 1435. The power board 1420 comprises a Wi-Fi module and is connected with an antenna 1430, an air-puff generator unit 1440 and an eye drop sub system 1445. The optical sensor board 1425 is also connected with the air-puff generator unit 1440 and the eye drop sub system 1445. The Optical sensor board 1425 connects the following modules: line-scan sensor 1450, LEDs 1455, video camera 1460, pattern projector 1465 and the air-puff pressure sensor 1470. Moreover, board 1425 is the sub-mount for all the electro-optics and optics components of the system.

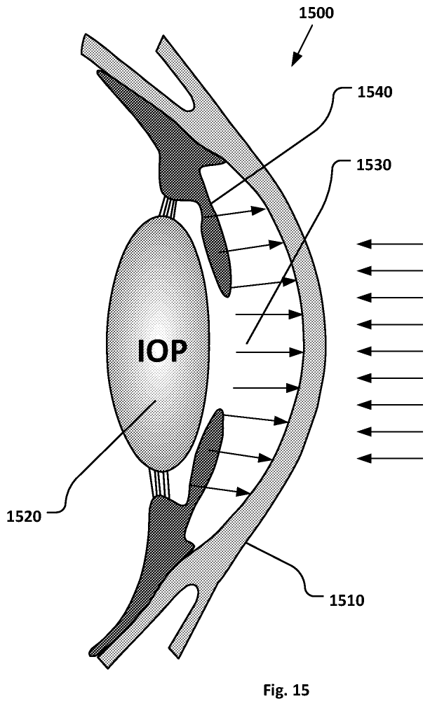

FIG. 15 shows an illustration of an eye 1500 comprising a cornea 1510 under intraocular pressure and air pressure, eye lens 1520, pupil 1530 and iris 1540. During measurement, an air pulse is delivered to the eye, which is in its initial state (regular state). The air causes the cornea to move inward and outwards to its initial curvature, passing through the applanated state twice. The whole process lasts approximately 30 ms to 50 ms for each measurement.

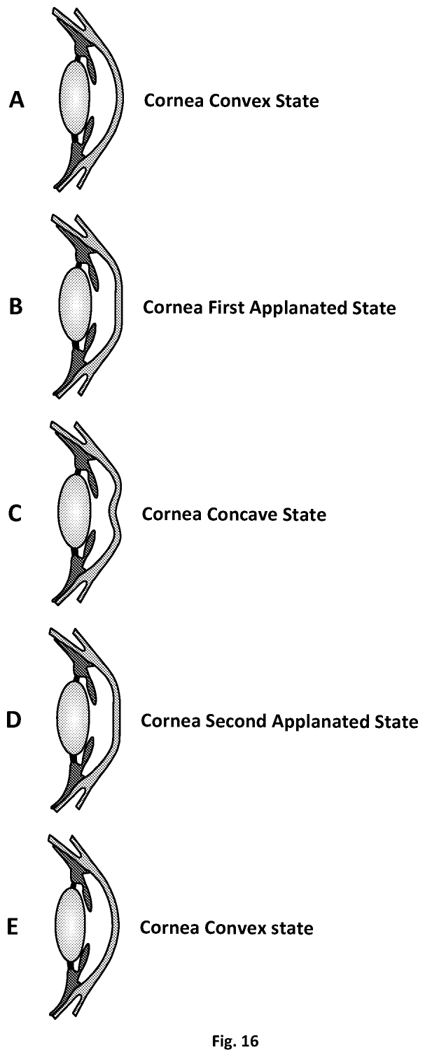

FIG. 16, A to E shows a corneal deformation cycle caused by the air puff.

A shows the cornea in its original and natural convex state.

B shows the cornea in a first state of applanation as the cornea is pushed inwardly by the air puff.

C shows the cornea in a concave state as the air puff pushes the corneal tissue beyond flat state shown in B.

D shows the cornea in a second state of applanation as the cornea returns.

E shows the cornea returns to its original and natural convex state.

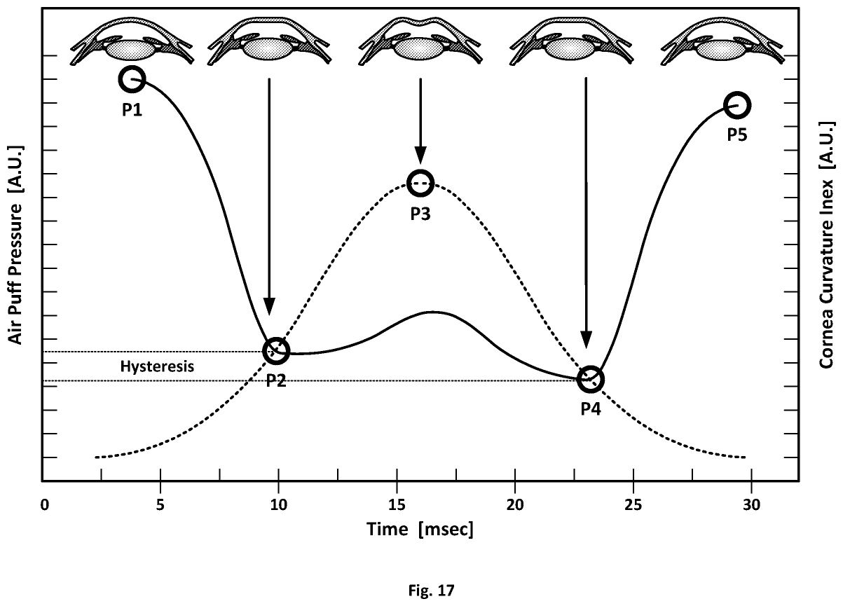

FIG. 17 shows a schematic graph of the air-puff pressure signal measured by the pressure sensor (1155A or 1155B of FIG. 11 or 260 of FIG. 12) and is characterized by a Gaussian bell curve shape. It is preferable to adjust the parameters of the air-puff mechanism to provide a pressure signal that is at least approximately symmetrical about a moment in time and has a suitable spread. The cornea curvature index (CCI) is measured and calculated in parallel to the air-puff pressure measurement. The CCI represents the cornea curvature and the minimal value associated with the cornea applanation point, with respect to FIG. 16. Point P1 is related to the cornea in its original and natural convex state.

Point P2 is related to the cornea in a first state of applanation as the cornea is pushed inwardly by the air puff. Point P3 is related to the cornea in a concave state. Point P4 is related to the second state of applanation. Point P5 is related to the cornea in its original and natural convex state. The two applanation points P2 and P4 are measured at two different pressures, this corneal hysteresis associated with the IOP measurement is found by calculating a pressure difference between the inward applanation pressure P2 and the outward applanation pressure P4.

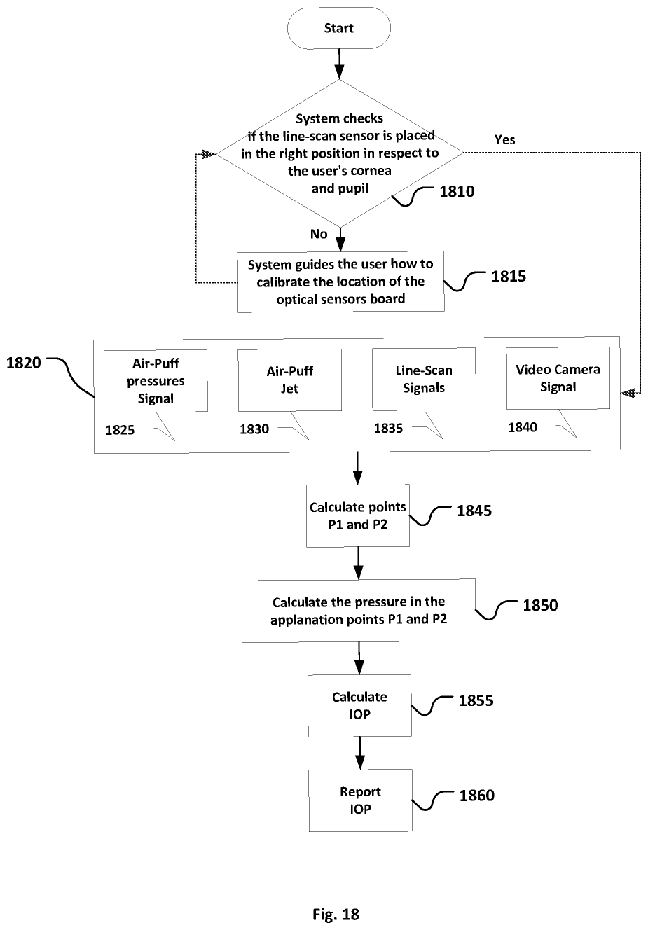

FIG. 18 is a flow chart showing the IOP measurement sequence carried out by the IOP non-contact tonometer system, according to embodiments of the present invention. It will be appreciated that the process may be done with one optical sensors board or two optical sensors boards. The process is described in relation to the optical sensors board. The process may be started when the user wears the head-mounted IOP unit 110 of the present invention or alternatively, when the user places his head in a fixed IOP unit. In step 1810, the system checks, using the video camera, if the line-scan sensor is placed in the right position in respect to the user's cornea and pupil. If it is, the process continues to step 1820. If it is not, in step 1815, the system guides the user how to calibrate the location of the optical sensors board by, for example, voice commands and the process returns to step 1810. In step 1820, the IOP measurement starts with four parallel and synchronized steps 1825 to 1840. In 1825, the system measures the air-puff pressure signal. In 1830, the air-puff mechanism releases the air puff. In 1835, the line-scan sensor records the cornea applanation. In 1840, the video camera records the cornea location. It will be appreciated that step 1820 may be done once or a number of times for achieving better results. In step 1845, the system calculates the two applanation points (P2 and P4 of FIG. 17) from the recorded data with respect to the air-puff pressure. In step 1850, the system calculates the pressure in the applanation points P1 and P2 as a function of the distance of the line-scan sensor from the cornea, cornea thickness, temperature, etc. In step 1855, the system calculates the IOP level. In step 1860, the system reports the IOP level.

As mentioned above in FIG. 13, the system of the present invention may further comprise an eye drop dispensing sub system. According to embodiment of the present invention, the eye drop dispensing sub system may dispense eye drops according to the measurements results or according to the user's need.

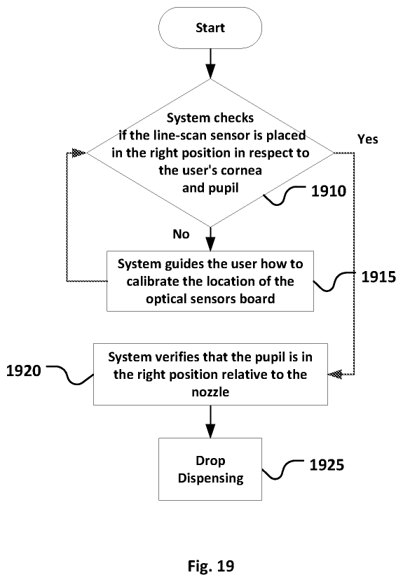

FIG. 19 is a flow chart showing the eye drop dispensing sequence, according to embodiments of the present invention. The process may be started when the user wears the head-mounted IOP unit 110 of the present invention or alternatively, when the user places his head in a fixed IOP unit. In step 1910, the system checks, using the video camera, if the line-scan sensor is placed in the right position in respect to the user's cornea and pupil. If it is, the process continues to step 1920. If it is not, in step 1915, the system guides the user how to calibrate the location of the optical sensors board by, for example, voice commands and the process returns to step 1910. In step 1920, the eye drop dispensing starts with verifying that the pupil is in the right position relative to the nozzle. In step 1925, an eye drop is released into the user's eye.

It will be appreciated that if the eye drop dispensing process is performed sequentially after the process described in conjunction with FIG. 18, step 1860 of FIG. 18 may be followed directly by step 1920 of FIG. 19.

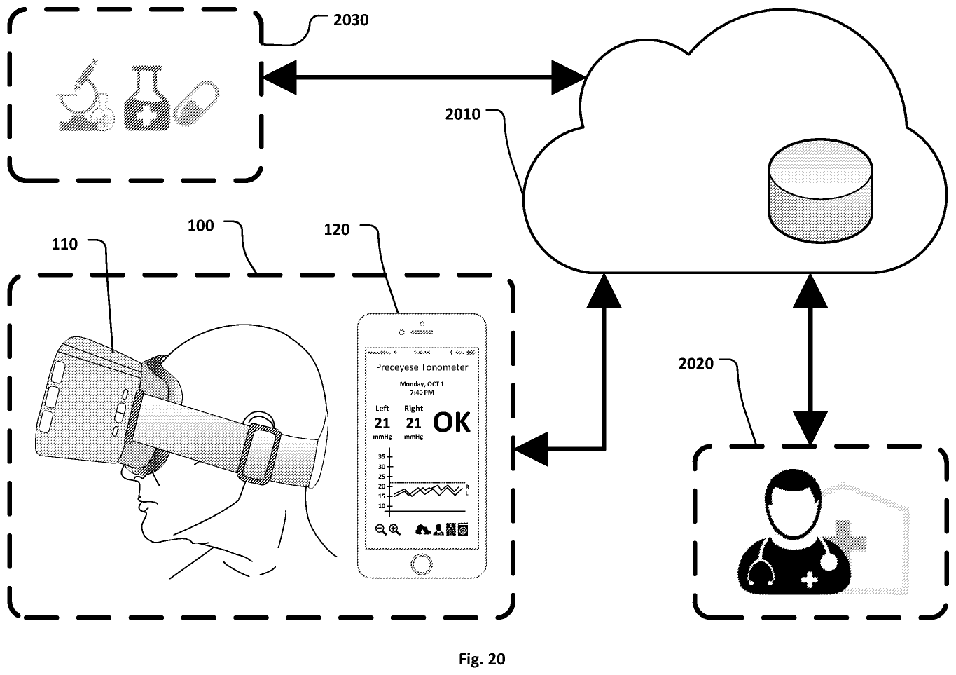

FIG. 20 shows the home-tonometry system 100 cloud base data flow. As described above, the IOP unit 110 is connected wirely or wirelessly to an electronic communication device such as a PC, tablet, smartphone 120, etc. The electronic communication device runs a user application for displaying the IOP results and is connected to a database (e.g., a cloud base database 2010). The patient's ophthalmologist or family physician 2020 may connect to user application and the data base 2010, with the patient's permission, and view the patient's IOP levels and the additional data. Additionally, the date base may be used by other users, with different authorization levels, like pharmaceutical companies or research institutes 2030.

The user application may show the measured IOP level of the last measurement and an accumulation graph of all the measures over time. In addition, the application may comprise a system built-in test (BIT) for checking initial condition(s) before measurement, user's personal information, user ophthalmologist, etc. This application is connected to a database that collects all the data from all users.

It will be appreciated by persons skilled in the art that the present invention is not limited to what has been particularly shown and described hereinabove. Rather the scope of the present invention is defined by the appended claims and includes combinations and sub-combinations of the various features described hereinabove as well as variations and modifications thereof which would occur to persons skilled in the art upon reading the foregoing description.

1. An ophthalmological analysis system, comprising:
an air-puff generating device configured to apply an air-puff to a user's at least one eye; and
at least one sensors board configured to detect and record deformation of a cornea of said user during said air-puff;
wherein said detection is a three dimensional (3D) active stereo detection;
said sensors board further configured to find two applanation points and hysteresis of said cornea and to calculate intraocular pressure in said user's at least one eye accordingly.

2. The system of claim 1, wherein said air-puff generating device comprises:
a piston, activated by automatic drive means, slidably received by a cylinder housing and axially driven relative to said cylinder housing to compress air within a compression chamber defined by said cylinder housing; and
an air discharge tube connected with said compression chamber for directing said air-puff along an optical axis towards said cornea.

3. The system of claim 2, wherein said automatic drive means comprise one of a linear motor, a rotary solenoid and a voice coil.

4. The system of claim 2, wherein said air-puff generating device further comprises a pressure sensor mounted inside said compression chamber.

5. The system of claim 2, wherein said air-puff is directed towards said cornea via an air-puff nozzle; wherein said air-puff generating device further comprises a pressure sensor mounted inside said air-puff nozzle.

6. The system of claim 1, wherein said air-puff generating device comprises:
a cylinder containing compressed air;
an air chamber connected with said cylinder via an electric valve; and
an air-puff nozzle connected with said air chamber via an electrical air regulator and an electrical valve.

7. The system of claim 6, wherein said air-puff generating device further comprises a pressure sensor mounted inside said air chamber.

8. The system of claim 6, wherein said air-puff generating device further comprises a pressure sensor mounted inside said air-puff nozzle;
said pressure sensor configured to measure said air-puff pressure over time.

9. The system of claim 1, further comprising an eye drop dispensing sub system comprising:
an eye drop reservoir connected with an electric dosage pump; and
a nozzle connected with said electric dosage pump;
said eye drop dispensing sub system configured to perform an eye drop cycle thereby allowing an accurate dosage and placement of at least one drop into said user's at least one eye.

10. The system of claim 9, wherein said eye drop reservoir is configured to dispense said eye drops as one of spray and aerosol.

11. The system of claim 1, wherein said at least one sensors board comprises:
a line-scan sensor;
wherein said line-scan sensor is divided into two viewing angles thereby allowing calculation of medial displacement over time;
an optical integrated lenses array mounted on top of the line-scan sensor;
a pattern-projector;
an air-puff nozzle;
a multi-spectral Light Emitting Diode (LED); and
a video camera.

12. The system of claim 11, wherein said optical integrated lenses array comprises four fixed mirrors.

13. The system of claim 11, wherein said optical integrated lenses array comprises two fixed mirrors and two movable mirrors.

14. A method of calculating intraocular pressure, comprising:
applying an air-puff to a user's at least one eye;
detecting and recording, using a three dimensional (3D) active stereo detection, deformation of a cornea of said user during said air-puff by a line-scan sensor divided into two viewing angles thereby allowing calculation of medial displacement over time;
finding two applanation points of said cornea; and
calculating intraocular pressure in said user's at least one eye accordingly.

15. The method of claim 14, wherein said applying an air-puff comprises:
activating, by automatic drive means, a piston inside a compression chamber thereby discharging said air-puff along an optical axis towards a cornea of said user.

16. The method of claim 15, wherein said automatic drive means comprise one of a linear motor, a rotary solenoid and a voice coil.

17. The method of claim 14, wherein said applying an air-puff comprises releasing a compressed air from a cylinder.

18. The method of claim 14, further comprising:
dispensing at least one eye drop into said user's at least one eye.
